(19) United States
(12) Patent Application Publication
(10) Pub. No.: US 2022/0115127 A1
(43) Pub. Date: Apr. 14, 2022

(54) METHODS AND APPARATUSES FOR TELEDENTISTRY

(71) Applicant: Sepand HOKMABADI, Tracy, CA (US)

(72) Inventor: Sepand HOKMABADI, Tracy, CA (US)

(21) Appl. No.: 17/499,664

(22) Filed: Oct. 12, 2021

Related U.S. Application Data
(60) Provisional application No. 63/090,133, filed on Oct. 9, 2020.

(51) Int. Cl. G16H 40/67; A61B 6/14; A61B 5/00; G16H 10/60; G16H 20/00; G16H 80/00; G16H 50/20

(52) U.S. Cl. CPC G16H 40/67; A61B 6/145; A61B 5/7465; G16H 50/20; G16H 20/00; G16H 80/00; G16H 10/60

(57) ABSTRACT

Provided herein are systems and methods for providing dental care to patients of a dental practice. The systems and methods can include virtual consultation hardware, software, and process flows that enable an expert dentist to focus solely on examining new and current patients, without the need to perform surgeries or in-person dental care. In one implementation, an expert dentist performs virtual consultations of new and current patients. The virtual consultation can include video conferencing, phone calls, texts, or emails. The expert dentist can review the patient's dental history, concerns, needs, and current diagnostics to prepare a treatment plan for the patient.

### CROSS REFERENCE TO RELATED APPLICATIONS

This application claims the benefit of U.S. Provisional Patent Application No. 63/090,133, filed Oct. 9, 2020, which is herein incorporated by reference in its entirety.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Dental schools focus primarily or exclusively on training Dental Surgeries and very little attention is paid to diagnosing. This lack of training on diagnosis leads to inconsistent and unpredictable recommendations from Dentist leads to patients having compromised oral health and poor experiences.

Traditional dental practices typically have one or more dentists to provide dental care to patients. The most experienced dentist in a dental practice (typically 10+ of experience at one location) can be referred to as an expert dentist. These expert dentists provide patients with better clinical outcomes due to better assessment of occlusal risk, periodontal risk, decay risk while spending more time truly understanding the patient's expectation. Since being in the same location for many years helps the expert dentist see the clinical results of their recommendations over a longer period of time, these expert dentists have more experience and a better understanding with long term clinical outcomes. Expert dentists also typically participate in continuing education beyond that needed for license renewal, which helps diagnose more advanced problems. Additionally, expert dentists often have more experience with technology to help identify problems earlier, such as digital X-rays, pantographic X-rays, intro-oral cameras, 3D tooth scanners, CBCT, etc. The physical limitation of a dental practice generally limits how much time, how many patients and in what geography the expert dentist can treat patients. For example, an expert dentist may be limited to seeing approximately 2,000 active patients, and accept up to only 50 new patients per month.

Traditional dental practices typically utilize a single location for all dental services, including new patient registration, initial exams, dental work, and hygiene visits. Referring to flowchart 100 of FIG. 1, a traditional dental diagnosing model includes, at block 102, bringing in new patients to the office or physical location (approximately 20-40 new patients per month depending on the number of dental practitioners). At block 104, the new patients typically arrive at the dental office and have a brief conversation to discuss dental health, concerns, prior treatment, insurance, etc. At block 106, these new patients typically are moved to an exam room in which they meet with a dentist (such as an expert dentist) for a brief exam and discussion about dental health and treatment planning. Next, at block 108, any dental surgery is performed by one of the dentists, and finally at block 110, the new patient is scheduled to receive routine preventative care dental hygiene appointments, typically twice per year for cleaning and evaluation. Since the expert dentist in a typical dental practice generally spends his or her time performing complicated procedures and surgeries, access to new and current patients for preventative care, diagnosis, and treatment planning is generally limited. The limitations of exams by the expert dentist leads to lowered standard of care in the industry. There is a need to increase access to expert dentists by the patient populations of traditional dental practices.

Traditional dental practices also typically use proprietary dental or patient management software that includes scheduling information, patient information, dental records, and any other type of data related to the practice or the dental care of the patients. These dental or patient management software suites are typically not customizable and are not configured or designed to interface with other software systems. Numerous challenges arise in trying to increase access to expert dentists, including virtually, while maintaining communication and or functionality with traditional dental or patient management software.

### SUMMARY OF THE DISCLOSURE

Implementations address the need to improve access to care for patients, the safety of dental diagnosis, quality of dental care, access to expert dentists, and efficiency of dental practices. The present application addresses these and other technical problems by providing technical solutions and/or automated agents that register new dental patients, acquire patient dental history data and desired outcomes, provide video and/or teleconferencing between a patient and an expert dentist for diagnosis and treatment planning, and communications and scheduling systems within a dental practice for coordinating patient care.

In particular, described herein are methods and systems that may allow dental practices to operate more efficiently and in a cost-effective manner. For example, described herein are systems that may guide one or more dental practitioner, including dentist, dental technician, and the like, in pre-screening and processing of patients, providing information and prompts to the technician or dentist during various phases of patient care management, may assist in training the dentist, and may shepherd patients through a number of coordinated pre-treatment and treatment encounters. These methods and apparatuses (e.g., software, firmware, etc.) may be particularly effective at enhancing interaction with a larger number of patients than has previously been possible.

For example, described herein are methods and systems configured to coordinate one or more dental professionals (e.g., auxiliaries, such as dental technicians, interviewers, etc., junior dentists, and senior dentists) in working with, including transitioning between, individual patients. In particular, these methods and systems for performing them, may include initial patient screening, including scheduling, prompting one or more auxiliary in collecting patient information, such as X-rays, insurance information, patient medical history, dental history, etc. The methods and apparatuses may include one or more interactive screens for engaging the auxiliary and preparing to automatically assign and prepare patient information for review by a dentist (e.g., a senior dentist). For example, the method or system may include coordinating the review of patient X-rays for possible treatment, review of insurance, review of patient-reported concern(s), determine and/or review patient risk of cavities, gum disease and biting forces, review of medical history, and/or review of dental history. In particular, this information may be compiled into a compact presentation for rapid review. Information may be digitally stored and displayed, and may be annotated by the auxiliary, senior dentist, and/or junior dentist.

For example, during an initial patient encounter, a patient may be seen in a dental office and may initially be interviewed by an auxiliary. A patient file may be opened at the time of the first encounter, which may automatically both guide the auxiliary in collecting patient-specific encounter information, and may also automatically or semi-automatically schedule the next step in the patient treatment, such as an interview with an expert dentist. The Auxiliary may be trained and guided (e.g., by the systems described herein) in asking the patient specific questions, and may collect the resulting responses in a patient response database that is shared with the senior and junior dentists. For example, the auxiliary may collect and save imaging data (e.g., X-rays, scans, etc.) of the patient's teeth as part of this initial screening. The auxiliary may also collect the patient's medical history, insurance information and may be prompted (e.g., by the software) to inquire about the reasons for seeking dental care. As part of this initial engagement, the auxiliary may take X-rays or other imaging of the teeth. While processing the patient, the system may prompt and prepare a senior dentist, and may schedule an encounter (e.g., within a fixed period of time, such as one hour, 45 minutes, 30 minutes, etc.) with the patient. Multiple patients may be concurrently processed and the system may coordinate collection of patient data, assigning a senior dentist, and may contact and prepare each senior dentist.

The senior dentist may have one or (more commonly) more separate patients scheduled for remote consultation. The system may communicate the schedule and preparatory information (e.g., a compact or streamlined set of patient information). For example, patient's may be scheduled in 10 min increments at a first location; while at this first location, an auxiliary may perform testing on the patient's teeth, either before or in combination with a remotely located senior dentist. For example, the auxiliary may measure pockets, cracked teeth, etc.

The senior (or a junior) dentist may then review the patient's information and may be prompted and kept on time and target by the software during an encounter with the patient. For example, the senior dentist may, following the prompted review of the compiled/collected patient data (or an extracted version of the patient data), meet with the patient and may diagnose the patient, after determining patient concerns. The type of care to be provided may be characterized in one or more categories, such as type 1 (urgent care), type 2 (preventative care), type 3 (cosmetic care), etc. The system may categorize (e.g., the dentist may select from a menus of options) the type of care to be provided, which may further refine the treatment to be provided, including selectin the junior dentist to handle the case, and scheduling the next (treatment) patient encounter(s).

For example, described herein are methods for providing dental treatment to a patient with a dental treatment system, comprising the steps of: obtaining patient information relating to a patient; obtaining dental diagnostic information relating to a patient's dental health; initiating a virtual consultation between the patient and an expert dentist; obtaining a dental treatment plan from the expert dentist; and presenting the dental treatment plan to the patient.

A system as described herein may include one or more processors, and a memory coupled to the one or more processors, the memory configured to store computer-program instructions, that, when executed by the one or more processors, implement a computer-implemented method, the computer-implemented method comprising: obtaining patient information relating to a patient; obtaining dental diagnostic information relating to a patient's dental health; initiating a virtual consultation between the patient and an expert dentist; obtaining a dental treatment plan from the expert dentist; and presenting the dental treatment plan to the patient.

In some variations a method may be a computer implemented method, comprising inputting, into a scheduling engine of a dental treatment system, availability of one or more patients, on ore more dental practitioners, and one or more treatment rooms of a dental facility; generating, in the scheduling engine, a treatment schedule that includes in-person treatment of the one or more patients and virtual consultations between the one or more patients and one or more of the dental practitioners; obtaining, in a patient information engine of the dental treatment system, dental health information pertaining to one or more patients; based on the treatment schedule, initiating, in a communication engine, a virtual consultation between a patient and a dental practitioner; and presenting, in a communication system, a dental treatment plan to the patient.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a block diagram illustrating a traditional dental diagnosing model.

FIG. 2A is a diagram showing an example of a computing environment configured to digitally scan a dental arch and determine a post-treatment tooth position score.

FIG. 2B is a diagram showing an example of a patient information engine(s).

FIG. 2C is a diagram showing an example a scheduling engine(s).

FIG. 2D is a diagram showing an example of a communication engine(s).

FIG. 3 is a flowchart that illustrates how an expert dentist can virtually communicate with patients at multiple locations utilizing the computing environment of FIG. 2A.

FIG. 4 is a flowchart showing a method of providing dental care to one or more patients.

FIGS. 5A-5B are flowcharts showing methods of providing dental care to one or more patients.

FIG. 6 is a flowchart showing a method of providing dental care to one or more patients.

FIGS. 7A-7B illustrate embodiments of a virtual waiting room of a dental computing environment.

FIGS. 8A-8D illustrate additional views of a patient facing side of a dental computing environment.

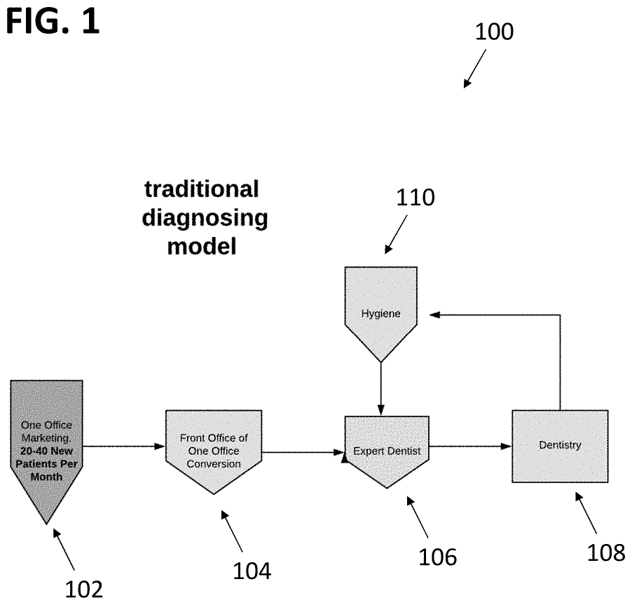

### DETAILED DESCRIPTION

Described herein are apparatuses (e.g., systems, computing device readable media, devices, etc.) and methods for improving the quality of dental diagnosis, dental care, access to expert dentists, and efficiency of dental practices. The present application addresses these and other technical problems by providing technical solutions and/or automated agents that register new dental patients, acquire patient dental history data and desired outcomes, provide video and/or teleconferencing between a patient and an expert dentist for diagnosis and treatment planning, and communications and scheduling systems within a dental practice for coordinating patient care. Apparatuses and methods described herein can provide a more uniform, higher standard of care, provide more accurate diagnosing and services, provide fewer dental complications, improve the overall patient experience, and vastly improve the profitability of a dental practice.

An “individual,” as used herein, may be any subject (e.g., human, non-human, adult, child, etc.) and may be alternatively and equivalently referred to herein as a “patient”, a “patient under treatment”, or a “subject.” A “patient,” as used herein, may but need not be a medical patient. An “individual” or a “patient,” as used herein, may include a person who receives dental treatment, including dental evaluations, dental cleanings, and dental surgery.

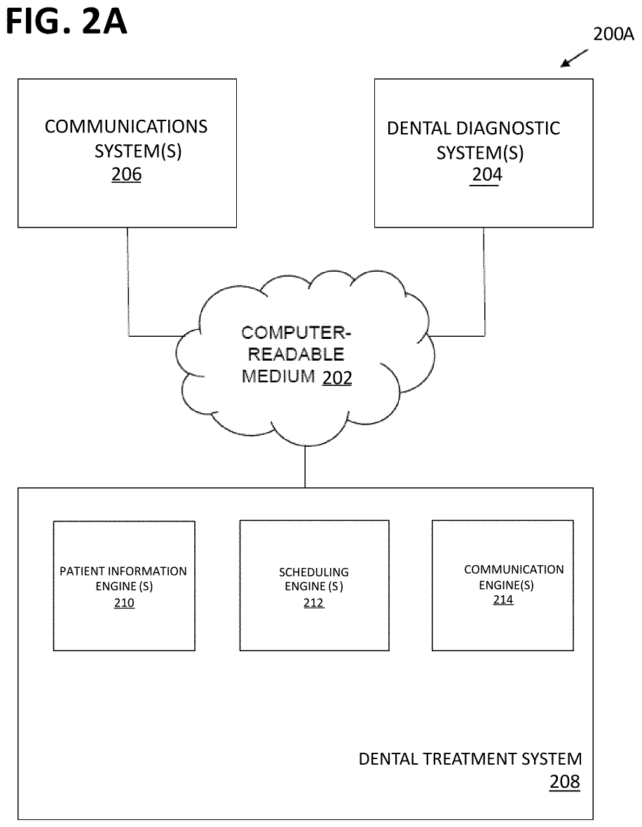

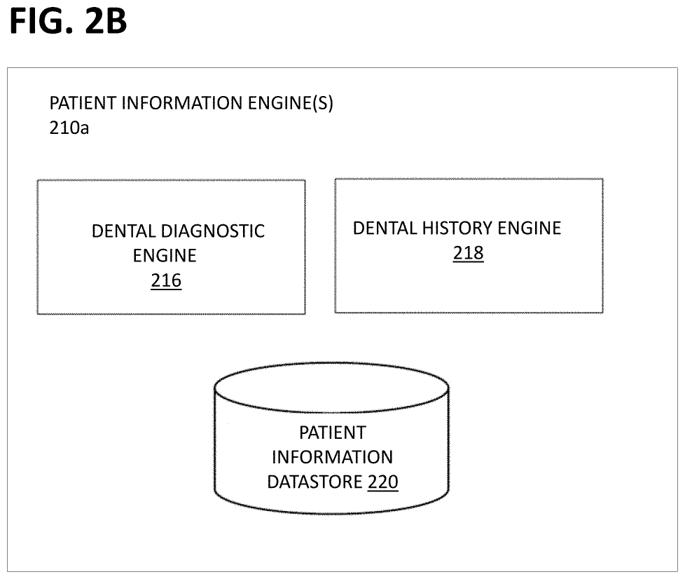

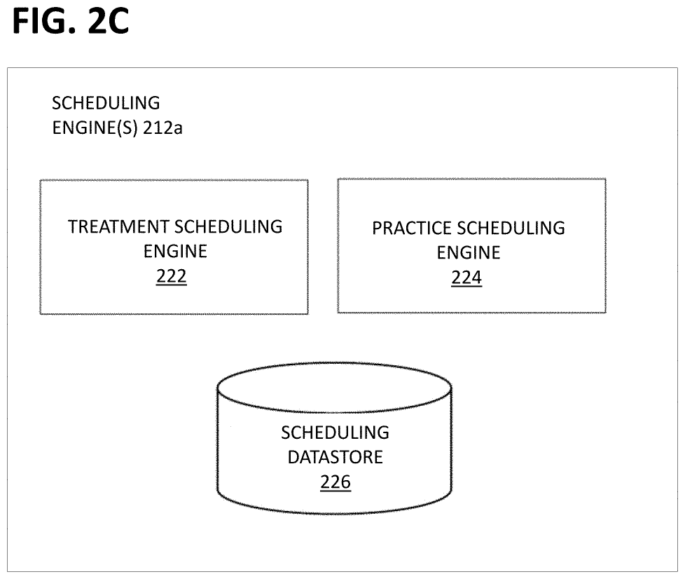

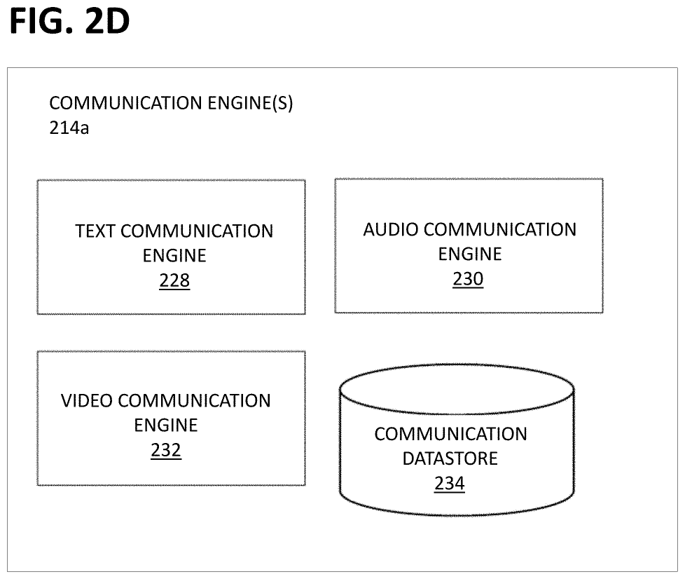

FIG. 2A is a diagram showing an example of a computing environment 200A configured to facilitate providing dental evaluations and services to one or more individuals. The environment 200A includes a computer-readable medium 202, a dental diagnostic system(s) 204, a communication system(s) 206, and a dental treatment system 208. One or more of the modules in the computing environment 200A may be coupled to one another or to modules not explicitly shown.

The computer-readable medium 202 and other computer readable media discussed herein are intended to represent a variety of potentially applicable technologies. For example, the computer-readable medium 202 can be used to form a network or part of a network. Where two components are co-located on a device, the computer-readable medium 202 can include a bus or other data conduit or plane. Where a first component is co-located on one device and a second component is located on a different device, the computer-readable medium 202 can include a wireless or wired back-end network or LAN. The computer-readable medium 202 can also encompass a relevant portion of a WAN or other network, if applicable.

The dental diagnostic system(s) 204 may include one or more computer systems configured for diagnosing dental problems or diseases, including, for example, X-ray imaging systems, radiograph instruments, intraoral cameras or scanners, infrared devices, caries detection systems, oral cancer and lesion screening systems, plaque detection systems, etc. A “dental arch,” as used herein, may include at least a portion of an individual's dentition formed by the individual's maxillary and/or mandibular teeth, when viewed from an occlusal perspective. A dental arch may include one or more maxillary or mandibular teeth of an individual, such as all teeth on the maxilla or mandible or an individual. The dental diagnostic system(s) 204 may include memory, one or more processors, and/or sensors to detect an individual's dental arch. In some implementations, the dental diagnostic system(s) 204 is configured to produce 2D or 3D scans or images of the individual's dentition.

The communications system(s) 206 may include one or more computer system configured to communicate audio, video, or text messages between two or more devices or systems. For example, the communications system(s) can comprise one or more personal computers, tablets, smartphones, fax machines, etc. The communications can be in the form of text messages, emails, audio messages, video messages, audible alerts, etc. The communications system(s) 206 may include memory, one or more processors, and a display device and/or audio device to display or announce the communication. The communications system(s) 206 may be implemented as part of a computer system, a display or speaker of tablet, smartphone, personal computer, etc.

The dental treatment system 208 may include one or modules including patient information engine(s) 210, scheduling engine(s) 212, and communication engine(s) 214. One or more of the modules of the dental treatment system 208 may be coupled to each other or to modules not shown. The dental treatment system 208 may include a computer system, including memory and one or more processors, configured to facilitate providing dental evaluations and services to one or more individuals. In some embodiments, the dental treatment system 208 can be implemented as software on a computer system. For example, the dental treatment system 208 can be implemented as an “app” or as software on one or more smartphones, tablets, personal computers, or the like. In one implementation, the patient information engine(s) 210 is configured to receive, store, and process patient information relating to an individual's dental health and history. In another implementation, the scheduling engine(s) 212 is configured to receive, store, and process individual schedules and availability of patients and/or dental practitioners, and is further configured to generate schedules and/or calendars coordinating the dental care of patients with the availability of dental practitioners, while accounting for space and equipment constraints of the dental practice (e.g., patient rooms, X-ray availability, etc.). In another implementation, the communication engine(s) 214 is configured to obtain as an input, generate, transmit, receive, and/or alert one or more communication devices with communications including text, audio, and/or video communications.

As used herein, any “engine” may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, “datastores” may include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

FIG. 2B is a diagram showing an example of a patient information engine(s) 210a. As described above, the patient information engine(s) can be a software module or component implemented within the dental treatment system (e.g., as software or an app). The patient information engine(s) 210a may include a dental diagnostic engine 216, a dental history engine 218, and a patient information datastore 220. One or more of the modules of the patient information engine(s) 210a may be coupled to each other or to modules not shown. The patient information engine(s) 210a of the dental treatment system 208 may implement automated agents to configured to process patient information from the dental diagnostic system(s) 204 and from other sources, including from direct input by a dental practitioner. The patient information can comprise, for example, dental insurance, personal information such as name, age, gender, etc. The patient information can further comprise prior dental history including prior dental treatments, prior dental X-rays or dental imaging, etc. The patient information can also comprise diagnostic information from the dental diagnostic system(s) 204, such as new/current dental imaging, scans, etc. Additionally, the patient information can comprise a new diagnosis or evaluation performed by a dental practitioner. The patient information can be stored on and received from a proprietary dental or patient management system. The patient information described herein can be automatically entered or transferred into the patient information engine(s) 210, or alternatively, can be manually input or entered by a dental practitioner.

The dental diagnostic engine 216 may implement one or more automated agents configured to receive, process, and/or generate dental diagnostic information relating to a patient's dental health. In one implementation, the dental diagnostic engine is configured to receive dental diagnostic information from the dental diagnostic system(s) 204. As described above, this dental diagnostic information can include X-ray imaging information, radiograph information, intraoral cameras or scanning information, infrared information, caries detection information, oral cancer and lesion screening information, plaque detection information, etc. The dental diagnostic engine 216 may also receive dental diagnostic information resulting from a dental examination by a dental practitioner. This dental diagnostic information can be entered automatically into the dental diagnostic information (e.g., from another dental or patient management software), or may be input manually by the dental practitioner. In some embodiments, the dental diagnostic engine is configured to automatically interface with the dental or patient management software.

The dental history engine 218 may implement one or more automated agents configured to receive, process, and/or generate prior dental history information. Prior dental history information can include, for example, prior dental treatment and/or diagnostic information including previous dental surgeries, dental treatments, and/or dental evaluations. In one implementation, the dental history engine 218 can implement one or more automated agents to obtain dental history information including, for example, personal information such as the patient's name, phone number, DOB, address, patient number, etc. that would allow for lookup of a prior treatment. The dental history information can be obtained by the dental history engine automatically (e.g., via communication with a dental database or other dental or patient management software), or alternatively, can be manually input by a dental practitioner.

The patient information datastore 220 may be configured to store patient information related to a patient's dental history or dental health. The patient information can be generated, accessed, or processed by the dental diagnostic engine 216 or dental history engine 218. The patient information may comprise the data or information described above, including dental diagnostic information and/or prior dental history information.

FIG. 2C is a diagram showing an example of a scheduling engine(s) 212a. As described above, the scheduling engine(s) can be a software module or component implemented within the dental treatment system (e.g., as software or an app). The scheduling engine(s) 212a may include a treatment scheduling engine 222, a practice scheduling engine 224, and a scheduling datastore 226. One or more of the modules of the patient scheduling engine(s) 212a may be coupled to each other or to modules not shown. The scheduling engine(s) 212a of the dental treatment system 208 may implement automated agents to configured to receive, process, and/or store scheduling information, including individual schedules and availability of patients, dental practitioners, physical room or equipment availability of a dental practice, and/or virtual waiting room or exam room availability and status. The scheduling information can comprise, for example, availability of individual patients to receive dental diagnostics or treatment at a dental practice. In another implementation, the scheduling information can comprise availability of dental practitioners, including dental assistants, dental hygienists, and dentists to evaluate, diagnose, treat, or communicate with patients either in person or virtually (e.g., by video call, phone call, or text/email messaging). The scheduling information can further comprise access to or availability of office space, evaluation rooms, surgical rooms, virtual waiting rooms or exam rooms, and diagnostic equipment of a dental practice. The scheduling information can be stored on and received from a proprietary dental or patient management system. In some embodiments, the treatment scheduling engine 222 is configured to update and/or manage the virtual waiting rooms of a dental practice, allowing providers such as dentists to view which patients are checked-in and waiting in a virtual waiting room. The scheduling information described herein can be automatically entered or transferred into the scheduling engine(s) 212a, or alternatively, can be manually input or entered by a dental practitioner.

The treatment scheduling engine 222 may implement one or more automated agents configured to receive, generate, and coordinate the availability of patients to receive dental care with the availability of dental practitioners to provide dental care. For example, the treatment scheduling engine 222 can receive, either automatically or by manual input, the availability of a patient. The treatment scheduling engine 222 can also receive, either automatically or by manual input, the availability of dental practitioners, including dental staff, dental hygienists, and dentists. The availability of patients and/or dental practitioners can account for vacation time, breaks, working schedules, etc. In some implementations, the treatment scheduling engine 222 can link or communicate with a scheduling database that can include one or more individual schedules of patient's and/or dental practitioners. The treatment scheduling engine 222 can determine availability of patients and/or dental practitioners based on scheduling information from the scheduling database. In other implementations, individual schedules and/or availability are input manually, such as by dental staff, into a scheduling software or database to coordinate schedules and availability. The scheduling information can be stored on and received from a proprietary dental or patient management system.

The practice scheduling engine 224 may implement one or more automated agents configured to receive, generate, and coordinate the availability of treatment rooms, virtual waiting rooms or exam rooms, surgical rooms, and/or dental diagnostic or treatment systems or hardware of a dental practice. In some implementations, the practice scheduling engine 224 can communicate with the treatment scheduling engine 222 to coordinate the availability of patients and/or dental practitioners with the availability of treatment rooms, virtual waiting rooms or exam rooms[, surgical rooms, and/or dental diagnostic or treatment systems or hardware For example, the practice scheduling engine 224 can receive, either automatically or by manual input, the availability of a patient for a physical visit to a dental facility or alternatively the availability and/or presence of the patient in a virtual waiting room. The treatment scheduling engine 222 can also receive, either automatically or by manual input, the availability of dental practitioners, including dental staff, dental hygienists, and dentists. The availability of patients and/or dental practitioners can account for vacation time, breaks, working schedules, etc. In some implementations, the practice scheduling engine 224 can link or communicate with a scheduling database that can include information related to the availability or usage of dental treatment rooms, dental surgical rooms, or dental equipment of a dental practice. In other implementations, availability of rooms or dental equipment are input manually, such as by dental staff, into a scheduling software or database to coordinate schedules and availability. The practice information can be stored on and received from a proprietary dental or patient management system.

The scheduling information datastore 226 may be configured to store scheduling information. As described above, the scheduling information can comprise, for example, availability of individual patients to receive dental diagnostics or treatment at a dental practice or in a virtual waiting room or virtual exam room. In another implementation, the scheduling information can comprise availability of dental practitioners, including dental assistants, dental hygienists, and dentists to evaluate, diagnose, treat, or communicate with patients. The scheduling information can further comprise access to or availability of office space, evaluation rooms, surgical rooms, and diagnostic equipment of a dental practice. The scheduling information can be generated, accessed, or processed by the treatment scheduling engine 222 or practice scheduling engine 224.

FIG. 2D is a diagram showing an example of a communication engine(s) 214a. The communication engine(s) 214a may include a text communication engine 228, an audio communication engine 230, a video communication engine 232, and a communication datastore 234. One or more of the modules of the communication engine(s) 214a may be coupled to each other or to modules not shown. The communication engine(s) 214a of the dental treatment system 208 may implement automated agents to configured to obtain as an input, generate, transmit, receive, and/or alert one or more communication devices with communications including text, audio, and/or video communications. The communication devices can include electronic devices not limited to personal computers, laptops, tablets, smartphones, fax machines, etc. The communications can include, for example, text messages, emails, voice messages, voice calls, video messages, video calls, faxes, visual alerts including blinking lights, and/or audible alerts such as beeps, alarms, or other audible notifications. In general, the communication engine(s) 214a facilitate communications between team members of a dental practice and patients of the dental practice, such as between a proprietary software or app loaded or installed on the patients' smartphones, tablets, or personal computers and between an app or software loaded or installed on the dental providers' smartphones, tablets, or personal computers.

The text communication engine 228 may implement one or more automated agents configured to receive, process, and/or generate text communications between one or more communication devices. The text communication engine can be implemented in an app loaded or installed on the patient's smartphone, tablet, or personal computer. In some implementations, the text communications are between dental providers or employees of a dental practice relating to the dental care, diagnosis, or treatment of one or more patients. In other implementations, the text communications are between dental providers and a patient relating to the dental care, diagnosis, or treatment of the patient. In one implementation, the text communication is simply a notification to a dental provider that a patient is ready to be examined either in-person or remotely (e.g., via audio or video communications in a virtual waiting room or exam room). In some implementations, the text communication can be in the form of text messages, email messages, etc. The text communications can be automatically generated by the text communication engine 228. For example, the text communication engine 228 of the communication engine(s) 214a may communicate with the scheduling engine(s) 212a, and may be configured to automatically generate a text communication when a patient is scheduled to receive dental treatment or diagnosis from a dental practitioner. In some implementations, the text communication can be sent to one or more dental practitioners or patients. In another implementation, the text communication engine 228 may communicate with the patient information engine(s) 210a, and may be configured to automatically generate a text communication to a dental provider with patient information including dental history, diagnosis, or a dental treatment plan.

The audio communication engine 230 may implement one or more automated agents configured to receive, process, and/or generate audio communications between one or more communication devices. The audio communication engine can be implemented in an app loaded or installed on the patient's smartphone, tablet, or personal computer. In some implementations, the audio communications are between dental providers or employees of a dental practice relating to the dental care, diagnosis, or treatment of one or more patients. In other implementations, the audio communications are between dental providers and a patient relating to the dental care, diagnosis, or treatment of the patient. In one implementation, the audio communication is simply an audible notification to a dental provider that a patient is ready to be examined either in-person or remotely (e.g., via audio or video communications). In some implementations, the audio communication can be in the form of audio messages, telephone calls, audible beeps or alerts, etc. The audio communications can be automatically generated by the audio communication engine 230. For example, the audio communication engine 230 of the communication engine(s) 214a may communicate with the scheduling engine(s) 212a, and may be configured to automatically generate an audio communication when a patient is scheduled to receive dental treatment or diagnosis from a dental practitioner. In some implementations, the audio communication can be sent to one or more dental practitioners or patients. In another implementation, the audio communication engine 230 may communicate with the patient information engine(s) 210a, and may be configured to automatically generate an audio communication to a dental provider with patient information including dental history, diagnosis, or a dental treatment plan.

The video communication engine 232 may implement one or more automated agents configured to receive, process, and/or generate video communications between one or more communication devices. The video communication engine can be implemented in an app loaded or installed on the patient's smartphone, tablet, or personal computer. In some implementations, the video communications are between dental providers or employees of a dental practice relating to the dental care, diagnosis, or treatment of one or more patients. In other implementations, the video communications are between dental providers and a patient relating to the dental care, diagnosis, or treatment of the patient. In one implementation, the video communication is simply a notification to a dental provider that a patient is ready to be examined either in-person or remotely (e.g., via audio or video communications). In another implementation, the video communication engine is implemented as a virtual waiting room. The provider(s) of a dental practice can interact with a software implementation of the video communication engine to see a list of scheduled patient appointments along with an indication if individual patients have “checked-in” virtually for their virtual consultation. In this embodiment, the provider(s) will be able to see the patients that are checked-in and waiting examination. Furthermore, the provider(s) can select or interact with the checked-in patients (e.g., such as by selecting, touching, or clicking a software interface associated with the patient) to load into a video or virtual call or meeting with the patient that is private and separate from the virtual waiting room (e.g., a virtual examination room). In some implementations, the video communication can be in the form of video messages, videos of the patient's dental anatomy or of dental scans, etc. The video communications can be automatically generated by the video communication engine 232. For example, the video communication engine 232 of the communication engine(s) 214a may communicate with the scheduling engine(s) 212a, and may be configured to automatically generate a video communication when a patient is scheduled to receive dental treatment or diagnosis from a dental practitioner. In some implementations, the video communication can be sent to one or more dental practitioners or patients. In another implementation, the video communication engine 232 may communicate with the patient information engine(s) 210a, and may be configured to automatically generate a video communication to a dental provider with patient information including dental history, diagnosis, or a dental treatment plan.

As described above, in some implementations, the video communication engine 232 can implement a virtual waiting room which queues patients waiting to have a virtual consultation with an expert dentist or other dental provider. The virtual waiting room can be, for example, a first-come first-served virtual waiting room in which patients are queued according to the time at which they enter the waiting room. Alternatively, the virtual waiting room can be queued according to previously scheduled dental appointments, and the provider(s) can view which patient's have checked-in or are available for consultation. Information can be communicated to each patient regarding an approximate wait time and/or queue number while they are waiting for the virtual consultation. In one embodiment, the expert dentist can pull individual patients from the queue for the virtual consultation. Patients can be pulled automatically according to their queue number, or in other implementations the expert dentist can choose to speak to a chosen individual patient. When the provider(s) pull a patient, the patient and the provider(s) or expert dentist are moved to a private virtual communication, such as a private video call, phone call, or the like.

The communication datastore 234 may be configured to store communication information related to communications between dental providers or between a patient and a dental provider. The communication information can be generated, accessed, or processed by the text communication engine 228, the audio communication engine 230, or the video communication engine 232. In some implementations, the communications can be stored on the cloud, such as in the computer readable medium 202.

Systems and methods are provided herein that enable a dental practitioner, such as an expert dentist, to see, speak to, communicate with, diagnose, and treat more patients than would be possible under a traditional dental care model. Additionally, systems and methods are provided herein that enable the dental practitioner, such as the expert dentist, to spend more time with each patient, facilitating better diagnosis, treatment planning, and care than is possible under a traditional dental care model.

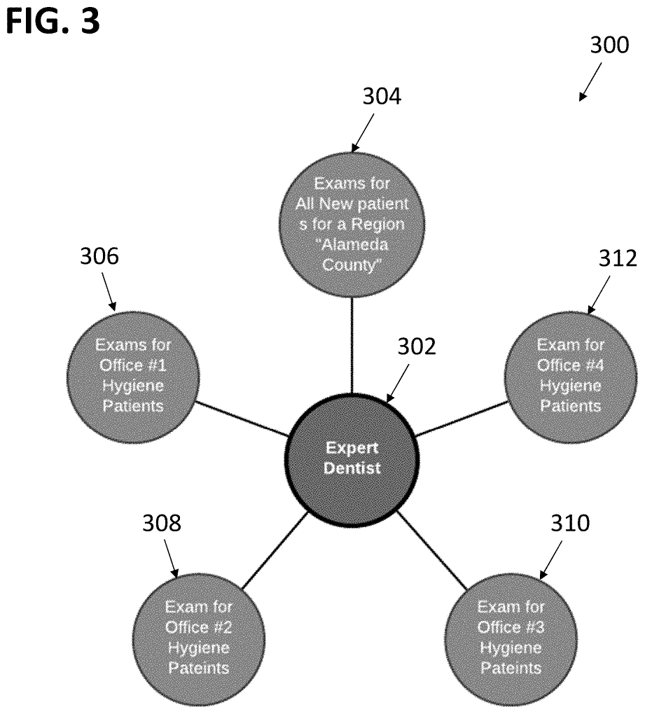

Referring to FIG. 3, a diagram 300 is shown that illustrates how an expert dentist at block 302 can virtually communicate with patients at multiple locations (shown by blocks 304-312) utilizing the system described above. For example, with the use of dental treatment system 208 of FIG. 2A, the expert dentist can perform teledentistry, via text communication, audio communication, and/or video communication, with patients at more than one physical location. Unlike traditional dental models, an expert dentist according to the embodiments provided herein does not perform dental surgeries allowing the expert dentist to focus solely on examining new patients and hygiene patients. Referring back to FIG. 3, the expert dentist can perform remote exams, diagnosis, and dental treatment planning throughout the course of the day with new patients from a registration or new patient center 304, and with hygiene patients from one or more dental offices, such as dental offices 306, 308, 310, and 312. The patients at each of the physical offices may receive treatment at that physical location from an on-site provider, such as dental cleanings, X-rays or other imaging, physical exams, and/or dental surgeries. Before or after the physical care is provided, the patients at those locations can also enter or queue into a virtual waiting room to be seen or consulted by the expert dentist. In some embodiments, the patients install an app or software on their smartphone, and enter the virtual waiting room through the app or software. When a specific patient is pulled from the virtual waiting room queue, the patient is connected into a one on one virtual call or video call with the expert dentist.

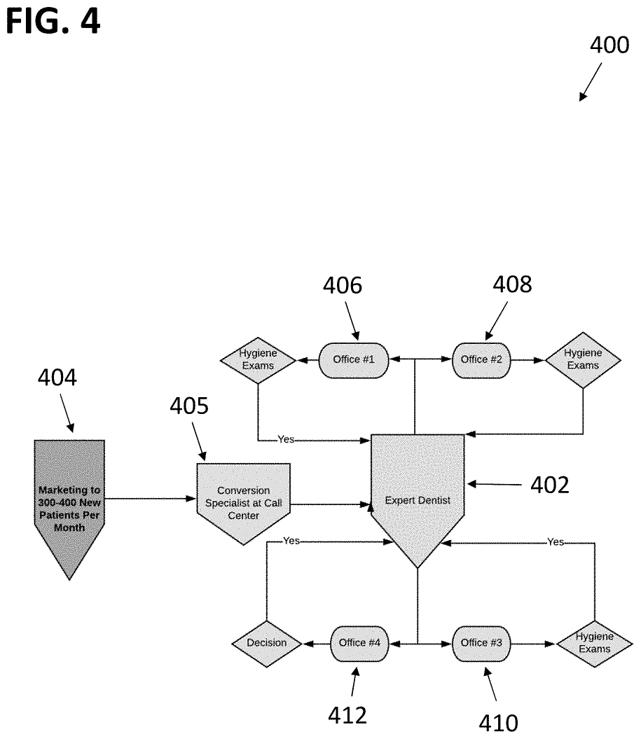

FIG. 4 is a flowchart 400 that illustrates how the systems and methods described herein expand the scope of clinical care in a dental practice. The flowchart can include the use of the systems described herein, such as the computing environment 200A described above and in FIGS. 2A-2D. Similar to above, an expert dentist as illustrated by block 402 can focus solely on examining new patients from registration or new patient center 404 and one or more dental offices, such as offices 406, 408, 410, and 412.

A dental auxiliary, or conversation specialist, at block 405 can conduct new patient interviews, which can include discussions with the new patient about chief concerns with dental health, medical history, dental history, insurance coverage, custom needs, and/or obstacles to receiving dental care (e.g., financial, time constraints, phobias, etc.). In some implementations, the dental auxiliary can conduct the initial interview in person at the new patient center. Alternatively, in other implementations, the dental auxiliary can conduct the initial interview with the patient remotely, such as with the communication engine 214 of the dental treatment system 208. The results of this discussion with the patient can be input, either automatically or manually, into the computing environment 200A, such as into the patient information engine 210 of the dental treatment system 208. Additionally, the dental auxiliary can gather scheduling information or availability from the new patient, which can be input either manually or automatically into the computing environment 200A, such as into the scheduling engine 212 of the dental treatment system 208.

While new patients are being registered and interviewed at the new patient center, one or more dental practitioners, such as an expert dentist at block 402, can be evaluating, communicating with, diagnosing, and/or developing dental treatment plans for a plurality of patients at one or more physical locations. For example, a single expert dentist at block 402 can evaluate, diagnose, treat, and/or communicate remotely with one or more patients at dental offices 406, 408, 410, and 412. Since the expert dentist 402 is not physically present at all of these physical locations, the expert dentist can communicate with the patients remotely with audio communications, text communications, or video communication devices, as described above. For example, the expert dentist and the patient(s) can communicate via the computing environment 200A, such as with the communication engine(s) 214 of the dental treatment system 208, as described above. The communication can be, for example, via text messaging or email, via audio communications or phone calls, or via video conferencing.

Coordination of the communications between the patient(s) and the expert dentist can be managed by the computing environment 200A, such as by the scheduling engine(s) 212 of the dental treatment system 208. For example, the scheduling engine(s) 212 can generate a daily, weekly, or monthly schedule that provides appointments/time slots for communications between a dental practitioner, such as an expert dentist, and all the patients receiving treatment that day. The schedule can take into account the dental practitioners availability, the scheduled physical appointments of the patients, the type of in-person treatment the patients are scheduled to receive (e.g., cleaning, cavity filling, surgery, etc.), and the availability of physical dental offices and/or diagnostic/treatment equipment. As described above, in some implementations the expert dentist can receive an alert or notification from the computing environment 200A when a patient is ready to meet virtually with the expert dentist. The communication can be coordinated through a virtual waiting room in which patients who are ready to meet virtually with a provider can check-in to the waiting room or be entered into a queue, and the provider can select individual patients from the waiting room to perform a virtual examination (e.g., via video call through an app loaded on the patient's phone).

The patients at the physical office locations, such as dental offices 406, 408, 410, and 412, can receive in-person dental care from other dental practitioners prior to or after communicating with the expert dentist. For example, in one implementation, the expert dentist at block 402 can communicate with the patient(s) prior to the patient receiving a dental cleaning or other in-person dental treatment. In other implementations, the expert dentist can communicate with the patient after the patient receives a dental cleaning or other in-person dental treatment. In this example, the expert dentist can review any findings or notes resulting from the dental treatment, and can also review any new dental imaging or diagnostics taken during the course of the patient visit. For example, new X-ray imaging and dental cleaning notes can be transmitted to the expert dentist, who can review the diagnostic materials to discuss with the patient remotely. The expert dentist can also review any additional patient information via the computing environment 200A, such as from the patient information engine(s) 210 of the dental treatment system 208. The patient information can include, as described above, prior dental history, prior dental treatment plans, and/or prior dental imaging or diagnostics.

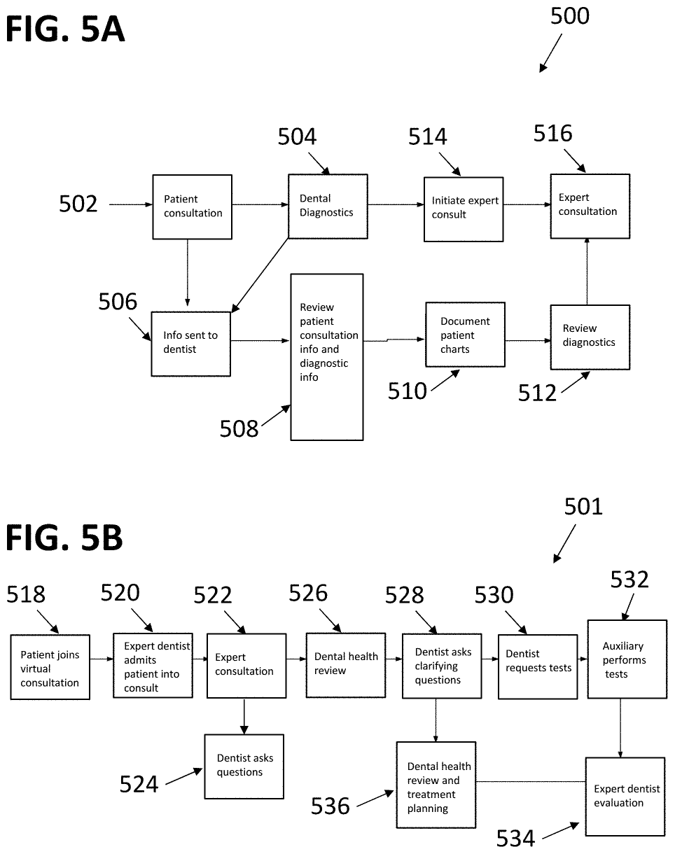

FIG. 5A is a flowchart 500 describing on implementation of a process flow for consulting with a dental patient. The dental patient can be a new dental patient (such as described above in block 405 of flowchart 400), or alternatively, the dental patient can be a current dental patient of the practice who is visiting a physical dental office for routine dental treatment such as dental hygiene visits or cavity fillings (as shown in blocks 406-412 of flowchart 400).

At block 502 of flowchart 500, a new dental patient arrives at a new patient center or welcome center (such as new patient center 404 of flowchart 400) or at a physical dental office (such as dental offices 406-412), and a dental practitioner or dental assistant conducts a patient consultation with the patient. For new patients, the consultation can be a new patient interview with the patient. The new patient interview can include a discussion of any health or dental concerns, obstacles to treatment, special needs, medical/social/dental history, insurance information, goals or hopes for dental treatment, etc. For current patients, the consultation can be a general questionnaire following up on the current state of the patient's dental health.

At block 504, a dental practitioner or dental staffer can perform diagnostics on the patient in person. This can include, for example, taking new X-ray photos, performing additional imaging of the patient's teeth, or performing other diagnostic tests or screening as needed.

Upon completion of the patient consultation and the dental diagnostics, information gathered during the consultation and during the dental diagnostics can be transmitted or sent to the expert dentist at block 506. In one implementation, the data is transmitted to the expert dentist with the computing environment 200A, such as the communication engine(s) 214 of dental treatment system 208. The information can be, for example, sent via text message, email, video message, etc. In other implementations, the expert dentist can be notified that new patient information is available for review, and the expert dentist can access a remote database containing the information, such as with a smartphone, tablet, or PC.

At block, 508, the expert dentist can review the information from the new dental patient consultation. As described above, the information from the new dental patient consultation can include information including patient concerns, social history, insurance history/coverage, medical history, dental history, custom requests, etc. At block 510, the expert dentist can update the patient's dental charts based on the information from the dental patient consultation. In tandem, at block 512, the expert dentist can review any diagnostics relating to the patient's dental health, such as new or prior X-ray imaging or any of the other diagnostic information mentioned above.

At block 514, the dental practitioner or dental auxiliary can initiate the expert consultation with the expert dentist. In one implementation, the expert consultation can be facilitated by communication engine(s) 214 of the dental treatment system 208, as described above. In one implementation, initiating the expert consultation comprises joining a virtual waiting room in which the patient waits for a virtual consultation with the expert dentist. The virtual waiting room can be, for example, a video conferencing wait room which displays a message to the patient with information estimating when the virtual consultation will begin. In some implementations, the virtual waiting room can be accessed with an electronic device, such as a smartphone, tablet, or PC. The virtual waiting room can access a camera and/or microphone of the patient's electronic device, and can display relevant information to the patient on a display or screen of the electronic device. For example, the virtual waiting room can provide an estimated wait time before the expert dentist will join the virtual consultation. In another embodiment, the virtual waiting room can comprise a queue of patients awaiting a virtual consultation with the expert dentist. The waiting room can be first-come-first-served and can include a queue number, or number in line for the virtual consultation. For example, if a particular patient is the fourth patient to enter the virtual waiting room, the virtual waiting room can convey to the patient that they are fourth in line to speak with the expert dentist.

Finally, at block 516, the expert dentist can join the patient in a virtual consultation. As described above, the virtual consultation can be a video call, an audio or phone call, or by text/email. When the expert dentist joins the virtual consultation, the patient can be pulled from the virtual waiting room into a virtual conference (e.g., a video conference) with the expert dentist. In embodiments where multiple patients are in the virtual waiting room, the queue can be updated to account for one of the patients being pulled into a virtual consultation with the dentist. In some embodiments, the expert dentist or provider can view the list of patients waiting in the virtual waiting room and select individual patients to consult with, which initiates a virtual or video call between the provider and the selected patient. As will be described in more detail below, the expert consultation with the expert dentist can include a discussion of the patient's goals for dental health, any concerns with past or current dental treatments, a review of patient information and dental diagnostics, discussion of dental treatment plans, and a discussion of future treatments and dental health.

FIG. 5B is a flowchart 501 describing on implementation of a process flow for an expert consultation between an expert dentist and a patient. The expert consultation can be between a new or current patient and the expert dentist, such as described above in block 516 of flowchart 500.

At block 518 of flowchart 501, the patient can join a virtual consultation. In some embodiments, joining the virtual consultation can comprise joining a virtual waiting room with other patients waiting for an expert dentist. The patient can join a virtual queue of other patients, and in some implementations an approximate wait time can be displayed or presented to the patient. In some implementations, the patient enters the virtual waiting room from a proprietary software or app loaded on the patient's smartphone, tablet, or PC. The patient's status or position can be displayed within the app.

At block 520 of flowchart 501, the expert dentist can admit a patient into a virtual consultation. In some embodiments, the expert dentist can load an app or software on a smartphone, tablet, or pc, and view the virtual waiting room from the app. The expert dentist can view the list of patients who are waiting in the virtual waiting room, and can individually select which patient to admit into the virtual consultation. As described above, the virtual consultation can be, for example, an audio call or a video call on an electronic device. In some embodiments, the virtual consultation is a video call viewable by both the expert dentist and the patient via an app or software loaded on their respective smartphones, tablets, or PCs. When the patient is admitted into the virtual consultation, the patient will be notified that the consultation is about to begin and the virtual connection is made between the patient and the expert dentist. For example, if the virtual consultation is a video consultation, then a video feed of the patient can be displayed on a screen of the expert dentist's device, and a video feed of the expert dentist can be displayed on a screen of the patient's device. An audio connection between the patient and the expert dentist can also be made. In some examples, a dental auxiliary such as a dental assistant, dental staffer, or dental hygienist can join the patient for the virtual consultation.

At block 522 of flowchart 501, the expert consultation can begin. First, at block 524, the expert dentist can ask the patient questions to build rapport with the patient and to put them at ease for the consultation. The questions can be personal in nature, small talk, etc.

Next, at block 526, the expert dentist can begin the dental health review of the patient. In one implementation, a dental auxiliary, dental hygienist, or dental staffer who is present with the patient can go over the patient's dental health case with the expert dentist. The dental auxiliary can present, for example, information relevant to the patient's dental health, including prior dental treatments, dental history, dental exam/diagnostic results, and past and present dental imaging. Alternatively, this information can be presented to the expert dentist directly through the smartphone/tablet/pc app or software.

Next, at block 528, the expert dentist can ask any clarifying questions of either the patient the dental auxiliary to better understand the patient's concerns/dental health. These follow up questions can be presented to the user over the virtual consultation through the app.

Next, at block 530, the expert dentist can request that the dental auxiliary perform one or more diagnostic or dental health tests on the patient. At block 532, the dental auxiliary can perform the diagnostic or dental health tests. These tests can include, but not be limited to, a cold test, bite stick test, probing, palpitation, decay probing, or EPT tests, as are commonly performed in a dental setting.

At block 532, the expert dentist can evaluate the patient's overall dental health, including evaluating the results of the tests performed at block 532 above. The evaluation can include an understanding of the patient's needs, concerns, and goals for dental health.

Finally, at block 534, the expert dentist can review the patient's dental health and discuss dental treatments that are needed, treatment options, etc. In some implementations, the expert dentist can present visuals relating to the patient's health to the patient. This can comprise, for example, sharing the dentist's screen to show the patient dental imaging or other diagnostics, including showing or explaining to the patient any potential courses of treatment. At the conclusion of the virtual consultation, the connection between the patient and the expert dentist can be terminated. In some implementations, the expert dentist is then queued into the next virtual consultation with subsequent patients based on the queue order of the virtual waiting room.

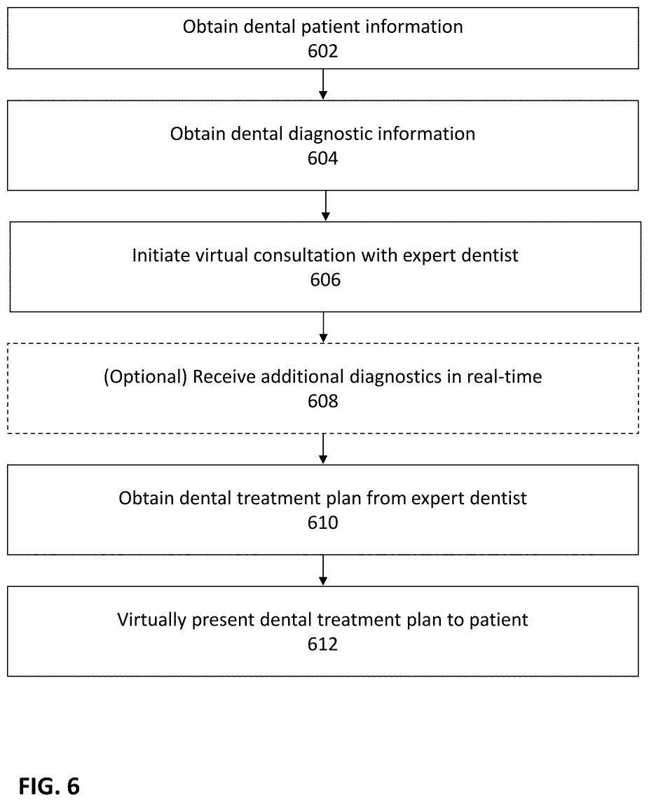

FIG. 6 is a flowchart 600 presenting a method for providing dental treatment to a patient. Flowchart 600 can incorporate the systems and methods described above, particularly the computing environment 200A of FIG. 2, and flowcharts 400, 500, and 501 of FIGS. 4, 5A, and 5B.

At step 602 of flowchart 600, the method can include obtaining dental patient information. The dental patient information can include, for example, concerns with dental health, medical history, dental history, insurance coverage, custom needs, and/or obstacles to receiving dental care (e.g., financial, time constraints, phobias, etc.). In one implementation, the dental patient information is entered into a dental treatment system, such as the dental treatment system 208 of computing environment 200A, described above. The data entry can be manual, or can be automatically entered into the system, such as from a dental or patient management software.

Next, at step 604 of flowchart 600, the method can include obtaining dental diagnostic information. The dental diagnostic information can be, for example, information from one or more computer systems configured for diagnosing dental problems or diseases, including, for example, X-ray imaging systems, radiograph instruments, intraoral cameras or scanners, infrared devices, caries detection systems, oral cancer and lesion screening systems, plaque detection systems, etc. In one implementation, the dental patient information is entered into a dental treatment system, such as the dental treatment system 208 of computing environment 200A, described above. The data entry can be manual, or can be automatically entered into the system such as from a dental or patient management software.

Next, at step 606 of flowchart 600, the method can including initiating a virtual consultation with an expert dentist. In some implementations, the virtual consultation is between a patient and the expert dentist, and can be facilitated by software or apps installed on one or more electronic devices such as smartphones, tablets, or personal computers. In one embodiment, the virtual consultation is a video conference call, in which the patient and expert dentist can see and speak to each other via the electronic devices. The virtual consultation can be initiated from a virtual waiting room, in which patients are automatically transferred to the expert dentist according to a queue, or alternatively, are individually selected by the expert dentist. During the virtual consultation, the expert dentist can discuss the patient's dental health, dental health history, concerns, needs, etc. to craft a dental treatment plan for the patient. The expert dentist can review the dental patient information and the dental diagnostic information to formulate the dental treatment plan.

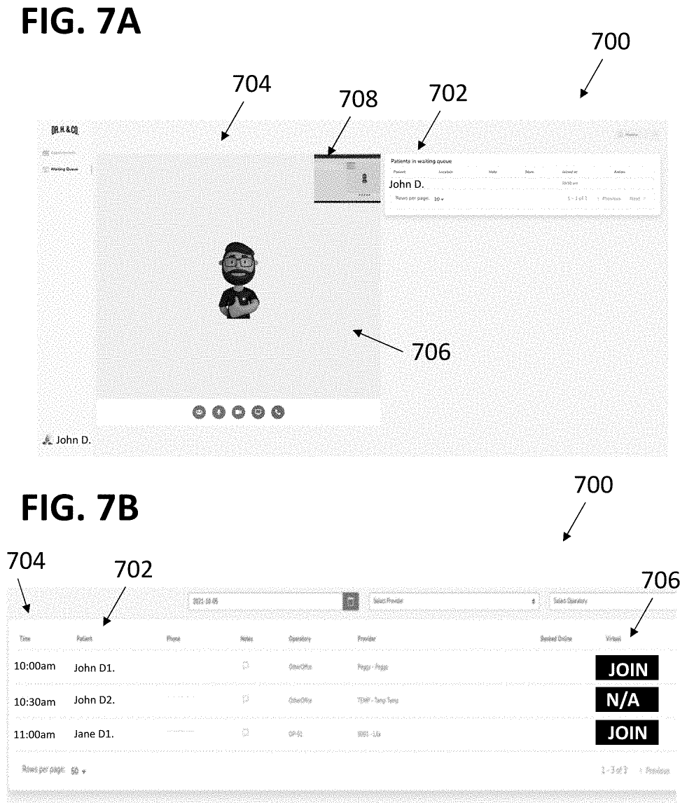

FIG. 7A illustrates one example or implementation of a virtual waiting room 700, which can be presented to a provider or expert dentist on a screen or device such as a smartphone, tablet, or PC. The virtual waiting room 700 can include a queue or waiting list 702, which can inform the provider of the patients that are in the virtual waiting room. In this example, the queue includes a single patient, John D. In some implementations, selecting a patient from the queue can initiate a video call or video conference 704 between the provider and the patient. In this example, a real-time video call of the patient 706 can be presented to the provider, and a video feed 708 of the provider can be shown which represents the main video feed that is shown to the patient on the patient's device.

FIG. 7B shows another example of a virtual waiting room 700 in which a plurality of patients 702 are shown listed in a queue according to a scheduled appointment time 704. In this embodiment, additional information can be provided in the waiting room such as each patient's phone number or contact information, the scheduled provider, and a virtual waiting room status 706 which indicates whether or not the patient has checked-in to the virtual waiting room. For example, referring to FIG. 7B, a provider can view the virtual waiting room and see which patients are in the waiting room and which patients are scheduled to join the waiting room. In this example, it can be seen that patients John D1. and Jane D1. are checked-in to the virtual waiting room. The provider or expert dentist can initiate a virtual call or exam with those patients by interacting with the “JOIN” button shown in the virtual waiting room status 706. In contrast, it can be seen that patient John D2. has not yet checked-in to the virtual waiting room, because the virtual waiting room status 706 shows that patient as “N/A” or not available. In this example, the provider can first virtually consult with patient John D1, and then can consult with patient Jane D1. since she is available and checked in. This enables the same efficiency in a virtual waiting room setting as would be found in a physical dental location where patients who are late or do not show up are skipped over for other patients in the physical waiting room.

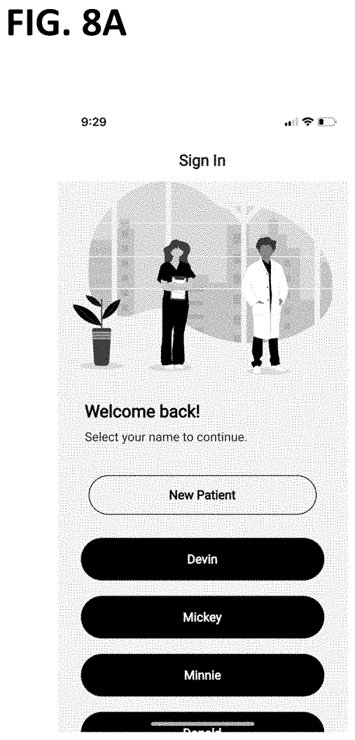

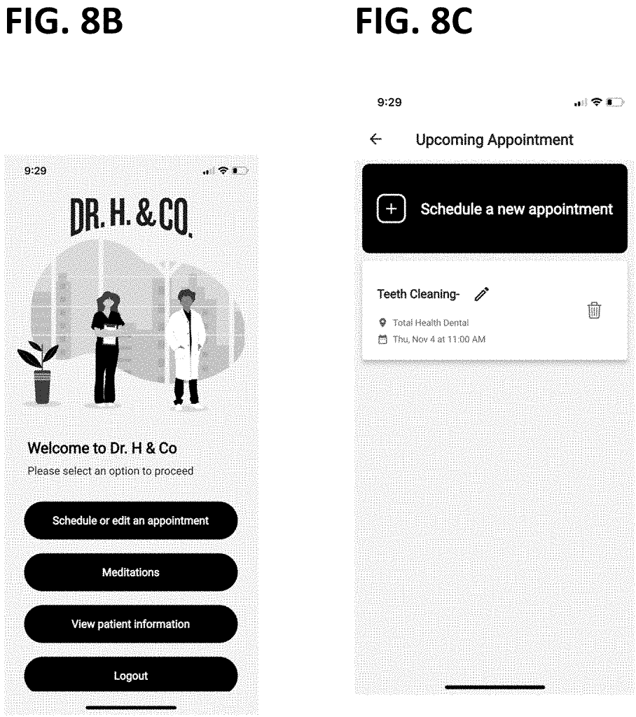

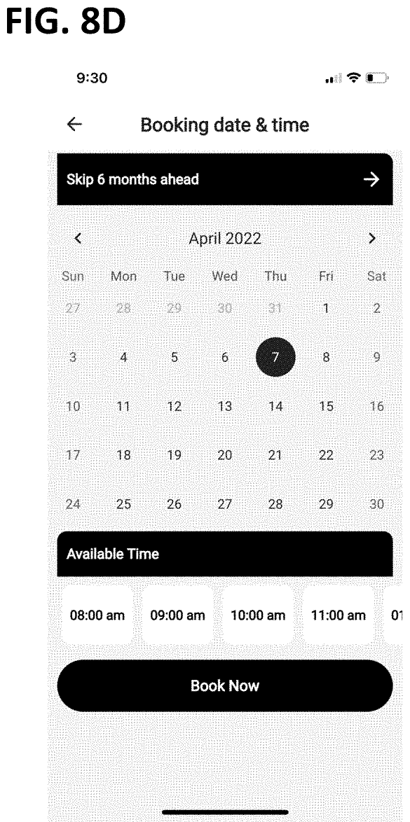

FIGS. 8A-8D illustrate some implementations of a patient facing dental app or software as previously described above. For example this software can be implanted as an app on a patient's smartphone, tablet, or PC. Referring to FIG. 8A, a sign-in screen can be presented to a patient in which the patient can identify as a new patient or as a previously registered patient. In some examples, multiple users can be pre-logged into the app, for example, allowing a family to manage the dental profiles of all patients within the family. Referring to FIG. 8B, upon logging into the app, the patient can view a selection of options, such as scheduling or editing an appointment, meditations, viewing patient information, or logging out. FIG. 8C illustrates an interface upon selecting “schedule or edit an appointment” in which the patient can view/edit existing appointments and FIG. 8D shows an interface within which the patient can schedule a new appointment.

Selecting the meditations option from the screen on FIG. 8B may provide the patient with relaxing sounds or images, or alternatively may provide the patient with entertainment (e.g., news, sports, tv, entertainment) while waiting for a virtual appointment. Selecting the “view patient information” option from the screen in FIG. 8B may present the patient with existing patient information, such as dental images, dental treatment history, dental treatment plans, etc.

In some embodiments, the expert dentist may optionally need additional information to formulate the dental treatment plan. Thus at optional step 608 of flowchart 600, additional diagnostics may be performed on the patient. In one implementation, a dental auxiliary, such as a dental hygienist, dental assistant, or dental staffer, may perform additional tests on the patient in-person. These tests can include, for example, a cold test, bite stick test, probing, palpitation, decay probing, or EPT tests. This additional testing information can be input in to the dental treatment system to be reviewed by the expert dentist, who can factor the additional testing information into the dental treatment plan.

At step 610 of flowchart 600, the dental treatment plan can be input into or obtained by the dental treatment system. For example, the expert dentist can manually input the dental treatment plan into software (e.g., updating a patient chart, ordering tests, ordering treatment, etc.) At step 612 of flowchart 600, the dental treatment plan can be presented to the patient. This can be, for example, via a screen share from the expert dentist on a video conference call, or by other digital or in-person communications, such as via an audio call, an email, text, or by presenting directly onto the patients device.

The method of flowchart 600 can be repeated throughout the day for all the patients of a dental practice, allowing an expert dentist to focus solely on providing expert care and consultations to a large number of patients, allowing the expert dentist to see more patients than would be possible under a traditional dental care model.

As a result of incorporating the systems and methods described herein, a welcome or new patient center can register up to 300-400 new patients per month (or more). The methods and apparatuses described herein allow an expert dentist to treat up to 8,000 active patients resulting in a broader range of patients that enjoy better clinical outcomes in the long term. Patients measurement of improved oral health can be measured through the average amount accepted per exam. The more the patient accepts per exam, the healthier their mouth is becoming. The less they accept, their existing problems continue to get worse. A typical average acceptance per examination for a dental practice is approximately $185 (e.g., if disease was detected and treatment for it was recommended, the patient average acceptance was $185 of treatment). However, implementing the methods and systems described herein can increase the average acceptance from approximately $185 to $300 which is a 62% increase per examination. The 62% increase in acceptance means patients oral health can be improved 62% more with the methods described herein vs a traditional method.

Various alternatives, modifications, and equivalents may be used in lieu of the above components. Although the final position of the teeth may be determined using computer-aided techniques, a user may move the teeth into their final positions by independently manipulating one or more teeth while satisfying the constraints of the prescription.

Additionally, the techniques described here may be implemented in hardware or software, or a combination of the two. The techniques may be implemented in computer programs executing on programmable computers that each includes a processor, a storage medium readable by the processor (including volatile and nonvolatile memory and/or storage elements), and suitable input and output devices. Program code is applied to data entered using an input device to perform the functions described and to generate output information. The output information is applied to one or more output devices.

Each program can be implemented in a high level procedural or object-oriented programming language to operate in conjunction with a computer system. However, the programs can be implemented in assembly or machine language, if desired. In any case, the language may be a compiled or interpreted language.

Each such computer program can be stored on a storage medium or device (e.g., CD-ROM, hard disk or magnetic diskette) that is readable by a general or special purpose programmable computer for configuring and operating the computer when the storage medium or device is read by the computer to perform the procedures described. The system also may be implemented as a computer-readable storage medium, configured with a computer program, where the storage medium so configured causes a computer to operate in a specific and predefined manner.

Thus, any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the invention and that methods and structures within the scope of these claims and their equivalents be covered thereby.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and/or methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the individual matter may be practiced. As mentioned, other embodiments may be utilized and derived there from, such that structural and logical substitutions and changes may be made without departing from the scope of this disclosure. Such embodiments of the inventive patient matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for providing dental treatment to a patient with a computer implemented dental treatment system, comprising the steps of:
obtaining patient information relating to a patient into the computer implemented dental treatment system;
obtaining dental diagnostic information relating to a patient's dental health into the computer implemented dental treatment system;
receiving an input in the computer implemented dental treatment system from an expert dentist to initiate a virtual consultation between the patient and the expert dentist;
obtaining in the computer implemented dental treatment system a dental treatment plan from the expert dentist; and
presenting the dental treatment plan to the patient.

2. The method of claim 1, wherein the patient information is selected from the group consisting of personal information, concerns with dental health, medical history, dental history, insurance coverage, custom needs, and obstacles to receiving dental care.

3. The method of claim 1, wherein the dental diagnostic information is selected from the group consisting of X-ray imaging, radiograph imaging, intraoral imaging, infrared imaging, caries detection results, oral cancer screening information, lesion screening information, and plaque detection information.

4. The method of claim 1, wherein initiating the virtual consultation comprises initiating a video conference between the patient and the expert dentist.

5. The method of claim 1, wherein initiating the virtual consultation comprises initiating an audio conference between the patient and the expert dentist.

6. The method of claim 1, further comprising receiving additional diagnostic information in real-time.

7. The method of claim 1, wherein the additional diagnostic information is selected from the group consisting of cold test information, bite stick test information, probing information, palpitation information, decay probing information, or EPT test information.

8. The method of claim 1, wherein the additional diagnostic information is obtained by a dental practitioner in the same physical location as the patient but remote from the expert dentist.

9. The method of claim 1, wherein obtaining the dental treatment plan comprises receiving the dental treatment plan as a manual input from the expert dentist.

10. The method of claim 1, wherein virtually presenting the dental treatment plan comprises displaying the dental treatment plan to the patient.

11. The method of claim 1, further comprising, prior to initiating the virtual consultation, placing the patient in a virtual waiting room.

12. The method of claim 11, wherein the virtual waiting room includes a plurality of patients awaiting a virtual consultation with the expert dentist.

13. The method of claim 12, wherein the plurality of patients are automatically selected for the virtual consultation in an order based on scheduled appointments.

14. The method of claim 12, wherein the plurality of patients are automatically selected for the virtual consultation in an order based on a first come first served basis.

15. The method of claim 1, wherein the method is implemented in an app loaded on a smartphone of the patient and a smartphone of the expert dentist.

16. A system comprising:
one or more processors;
memory coupled to the one or more processors, the memory configured to store computer-program instructions, that, when executed by the one or more processors, implement a computer-implemented method, the computer-implemented method comprising:
obtaining patient information relating to a patient;
obtaining dental diagnostic information relating to a patient's dental health;
initiating a virtual consultation between the patient and an expert dentist;
obtaining a dental treatment plan from the expert dentist; and
presenting the dental treatment plan to the patient.

17. A computer implemented method, comprising:
inputting, into a scheduling engine of a dental treatment system, availability of one or more patients, on ore more dental practitioners, and one or more treatment rooms of a dental facility;
generating, in the scheduling engine, a treatment schedule that includes in-person treatment of the one or more patients and virtual consultations between the one or more patients and one or more of the dental practitioners;
obtaining, in a patient information engine of the dental treatment system, dental health information pertaining to one or more patients;
based on the treatment schedule, initiating, in a communication engine, a virtual consultation between a patient and a dental practitioner; and
presenting, in a communication system, a dental treatment plan to the patient.

18. A method of providing dental care to one or more patients, comprising:
viewing a virtual waiting room implemented in an app on a smartphone, tablet, or PC, the virtual waiting room presenting a queue of one or more patients awaiting a virtual dental consultation;
selecting one of the patients from the queue to initiate a virtual consultation with the selected patient; and
providing a dental consultation to the selected patient virtually through the app.

19. The method of claim 19, wherein selecting further comprises selecting the patients according to a first come first served basis.

20. The method of claim 19, wherein selecting further comprises selecting the patients according to scheduled appointment times.

21. The method of claim 1, wherein obtaining the patient information further comprises obtaining patient information automatically from a dental practice management software.

22. The method of claim 1, wherein obtaining the dental diagnostic information further comprises obtaining dental diagnostic information automatically from a dental practice management software.

23. The method of claim 1, wherein receiving the input from the expert dentist further comprises receiving an input from a smartphone, tablet, or PC of the expert dentist to initiate the virtual consultation.

24. The method of claim 1, wherein obtaining the dental treatment plan further comprises obtaining audio and video recordings of the expert dentist with a smartphone, tablet, or PC of the expert dentist.

25. The method of claim 1, wherein presenting the dental treatment plan to the patient further comprises presenting audio and video recordings of the expert dentist to a smartphone, tablet, or PC of the patient.
